(19) United States
(12) Patent Application Publication
(10) Pub. No.: US 2008/0077046 A1
(43) Pub. Date: Mar. 27, 2008

(54) SNAP-OFF SPECIMEN COLLECTION DEVICE

(75) Inventor: Larry Burg, Framingham, MA (US)

Correspondence Address: Vista IP Law Group LLP, Suite D-2, 12930 Saratoga Avenue, Saratoga, CA, 95070, US

(73) Assignee: Cytyc Corporation, Marlborough, MA (US)

(21) Appl. No.: 11/467,068

(22) Filed: Aug. 24, 2006

(51) Int. Cl. A61B 10/00

(52) U.S. Cl. 600/569

(57) ABSTRACT

Disclosed herein is a cell collection device, comprising a shaft having a proximal portion and a distal portion, each portion having a proximal end and a distal end; a detachable joint between said proximal portion and said distal portion; and a cell collection portion at said distal end of said distal portion; whereby when said proximal portion and said distal portion are detached at said detachable joint, said proximal end of said distal portion is attachable to a vial cap.

### FIELD OF THE INVENTION

The present invention relates to the field of specimen collection devices, and in particular, devices directed to collect cells by scraping the cervix during the Pap test.

### BACKGROUND

Physicians commonly acquire certain clinical specimens by scraping or brushing the target tissue with a specimen collection device. One such application of this methodology is the Pap test, whereby cells of the cervix are obtained by scraping the ectocervix and endocervix with a collection device such as a spatula, brush, or cervical broom. These cells may be subsequently smeared onto a slide as in the conventional methodology or deposited into a liquid preservative solution as in the ThinPrep® Pap Test (TPPT, Cytyc Corp.) or SurePath® (TriPath Imaging, Inc.).

The above methods require the physician to swish or swirl the collection device in the preservative solution to remove the cells from the collection device. This step may lead to variable cell recoveries that are dependent on the physician's technique. A more vigorous and/or lengthy action is likely to result in greater cell recovery. A less diligent effort may lead to loss of cells and possibly an inadequate specimen due to insufficient cell concentration. Further, while swishing or swirling the collection device in the preservative solution, some of the solution may splash out of the vial and possibly on the physician's hands, leading to loss of cells and physician discomfort.

Therefore, there is a need in the art for a collection device that can consistently transfer most of the collected cells to the preservative solution, where such transfer is independent of physician technique, while reducing the chance for spills or splashes.

### SUMMARY OF THE INVENTION

In one embodiment, a cell collection device comprises a shaft having a proximal portion and a distal portion, each portion having a proximal end and a distal end; a detachable joint between said proximal portion and said distal portion; and a cell collection portion at said distal end of said distal portion; whereby when said proximal portion and said distal portion are detached at said detachable joint, said proximal end of said distal portion is attachable to a vial cap.

Other and further embodiments and features thereof will be apparent from the following detailed description, taken in conjunction with the drawings.

### BRIEF DESCRIPTION OF THE DRAWINGS

It will be appreciated that the drawings are not necessarily to scale, with emphasis instead being placed on illustrating the various aspects and features of embodiments of the invention, in which:

FIG. 1A depicts one embodiment of a cell collection device, a broom being shown, having a detachable joint.

FIG. 1B depicts the embodiment shown in FIG. 1A with the proximal and distal portions detached at the detachable joint.

FIG. 1C depicts one embodiment of a cell collection device, a broom being shown, having a detachable joint.

FIG. 1D depicts the embodiment shown in FIG. 1C with the proximal and distal portions detached at the detachable joint.

FIG. 2A depicts one embodiment of a cell collection device having a movable sheath at a resting position covering a detachable joint.

FIG. 2B depicts one embodiment of a cell collection device having a movable sheath, with the movable sheath in between a resting position covering the detachable joint and a resting position where the detachable joint is uncovered.

FIG. 2C depicts one embodiment of a cell collection device having a movable sheath at a resting position where the detachable joint is uncovered.

FIG. 3A depicts one embodiment of the vial cap with a centered female joint.

FIG. 3B depicts one embodiment of attaching a cell collection device to a vial cap having a centered female joint.

FIG. 3C depicts one embodiment of a curved or spiral groove on the female joint of a vial cap.

FIG. 3D depicts one embodiment of a straight groove with a 90 degree turn on the female joint of a vial cap.

FIG. 4A depicts one embodiment of the invention where a cell collection device is attached to a vial cap and is hanging within a vial.

FIG. 4B depicts one embodiment of the invention where a cell collection device is attached to a vial cap and is hanging within a vial at an angle.

FIG. 4C depicts one embodiment of the invention where a cell collection device is attached to a vial cap off-centered and is hanging within a vial.

FIG. 4D depicts one embodiment of the invention where two cell collection devices are attached to a vial cap and are hanging within a vial.

### DETAILED DESCRIPTION OF THE ILLUSTRATED EMBODIMENTS

Embodiments of the invention are described below. It is, however, expressly noted that the invention is not limited to these embodiments, which are shown for purposes of illustration and not limitation.

Certain embodiments of the invention are directed to a cell collection device having a detachable joint. The cell collection device of the present invention can be used for collecting cells from the body of an animal, such as a human patient or other mammals, such as from the cervix, urethra, mouth, etc.

The cell collection devices disclosed herein have the added advantage that the amount of cells recovered from a patient is independent of the diligence of the medical practitioner in swirling the device in the preservative solution. The device is inserted in the preservative solution shortly after the sample is taken and remains in the preservative solution until the sample reaches the laboratory. Once the vial containing the preservative solution and the collection device is capped, the sample can be shaken, either by hand or with a vortex device in a controlled, user-independent manner, either at the point where the sample is collected or in the laboratory where the sample is analyzed. This would ensure that most of the cells collected from the patient have been transferred to the preservative solution and are available for subsequent analysis. Furthermore, since the transfer of cells from the collection device to the preservative solution takes place in a closed vessel, there is virtually no risk that any of the solution, and therefore any of the cells, are lost and/or contaminate the hand of the medical practitioner.

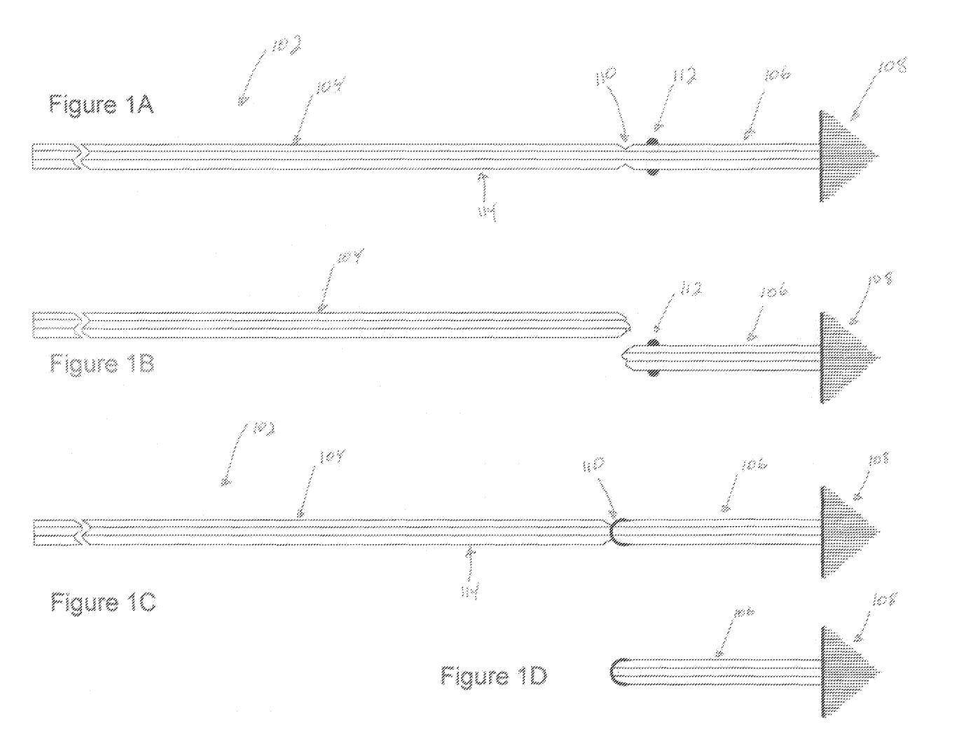

FIG. 1A shows one embodiment of a cell collection device disclosed herein. The cell collection device 102 consists of a shaft 114 and a cell collection portion 108. The shaft 114 comprises a proximal portion 104 and a distal portion 106. In the context of the present disclosure, the word “proximal” relates to the portion of the device closest to the hand of the physician and the word “distal” relates to any portion of the device closest to the cell collection portion 108.

The proximal portion 104 and the distal portion 106 are separated by a detachable joint 110. In the embodiment shown in FIG. 1A, the detachable joint is an area of the shaft 114 where the diameter of the shaft is narrower than the diameter of the shaft elsewhere. The narrow diameter creates a weak spot in the shaft 114, which enables the user of the device, such as a physician, to break the shaft 114 at the weak spot, thereby separating the proximal portion 104 from the distal portion 106. FIG. 1B shows an embodiment of the invention in which the detachable joint 110 is broken.

In other embodiments, the detachable joint 110 can comprise a male portion and female portion, in which the male portion is on the proximal portion 104 and the female portion is on the distal portion 106, or vice versa, i.e., the female portion is on the proximal portion 104 and the male portion is on the distal portion 106. Prior to collecting the sample from the patent, the male portion of the joint 110 and the female portion of the joint 110 are connected, which results in the proximal portion 104 and the distal portion 106 to be attached. Once the cells are collected, the physician simply moves the proximal portion 104 away from the distal portion 106, thereby separating the two portions.

In some embodiments, the male and female portions of the joint 110 are held together by friction lock. In other embodiments, the male female portions of the joint 110 comprise at least one nub on either the male or female portion and a corresponding groove on the other portion to ensure a tighter and stronger fit. In further embodiments, the male portion of the joint 110 screws into the female portion of the joint 110. In these embodiments, to separate the proximal portion 104 from the distal portion 106 the physician rotates one portion with respect to the other portion to unscrew the proximal portion 104 from the distal portion 106.

In some embodiments, the proximal end of the distal portion 106 comprises at least one nub 112, which can fit into a corresponding groove in the underside of a vial cap, as discussed in greater detail below.

In some embodiments, such as the one depicted in FIGS. 1C and 1D, the detachable joint 110 is designed such that when it is broken, the proximal end of the distal portion 106 consists of a ball end capable of making a ball and socket snap connection with a vial cap.

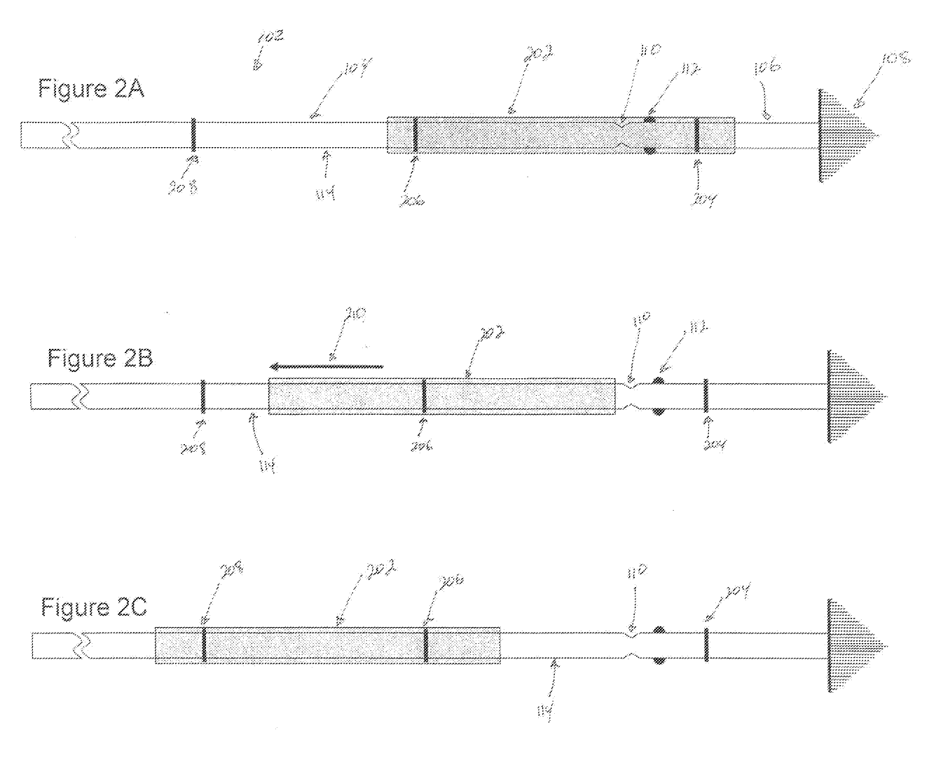

In some embodiments, the narrow diameter of the shaft 114 at the detachable joint 110 renders the shaft 114 too weak at this particular spot. There may therefore be a danger that the shaft 114 may break at the detachable joint spot 110 inside the patient, at which point the recovery of the distal portion 106 becomes difficult, or dangerous. To prevent such an outcome, the cell collection device 102 of these embodiments comprises a movable sheath 202, as shown in FIG. 2A.

In some embodiments, the movable sheath 202 is a hollow cylinder that fits over the shaft 114. The movable sheath 202 can slide from the distal end of the shaft 114 to the proximal end of the shaft 114 and back. The sheath 202 can rest in at least two positions: an extended position, as shown in FIG. 2A, which covers the detachable joint 110, and a retracted position, as shown in FIG. 2C, at which position the detachable joint 110 is uncovered.

Before inserting the collection device 102 inside a patient, the physician makes sure that the movable sheath 202 is in the extended position. In some embodiments, the shaft 114 comprises at least one nub or ridge 204, which in some embodiments creates a friction lock. In other embodiments, the nub or ridge 204 has a corresponding groove on the sheath 202, which creates a tighter lock. In some embodiments, there is a second nub or ridge 206 at the proximal end of the extended position to hold the sheath 202 in place more tightly.

After the physician has completed the collection of cells and has removed the cell collection device 102 from the patient, the physician slides the sheath 202 in the direction 210, FIG. 2B, towards the proximal end of the device 102. The sheath 202 comes to rest at the retracted position, shown in FIG. 2C. In some embodiments, the nub or ridge 206 fits into the groove that fit over the nub or ridge 204 in the extended position. In other embodiments, the shaft 114 comprises a third nub or ridge 208, which fits into the groove that fit over the nub or ridge 206 in the extended position.

In some embodiments, the shaft 114 comprises only one nub or ridge 206. The nub or ridge 206 fits into a groove at the proximal end of the sheath 202 in the extended position and fits into a groove at the distal end of the sheath 202 in the retracted position. In other embodiments, the shaft 114 comprises two nubs or ridges 204 and 208. Nub or ridge 204 fits into a groove at the distal end of the sheath 202 in the extended position, while nub or ridge 208 fits into a groove at the proximal end of the sheath 202 in the retracted position. In other embodiments, the shaft 114 comprises three nubs or ridges 204, 206, and 208. Nub or ridge 204 fits into a groove at the distal end of the sheath 202 and nub or ridge 206 fits into a groove at the proximal end of the sheath 202 in the extended position, while nub or ridge 208 fits into a groove at the proximal end of the sheath 202 and nub or ridge 206 fits into a groove at the distal end of the sheath 202 in the retracted position. In further embodiments, the shaft 114 comprises additional nubs or ridges.

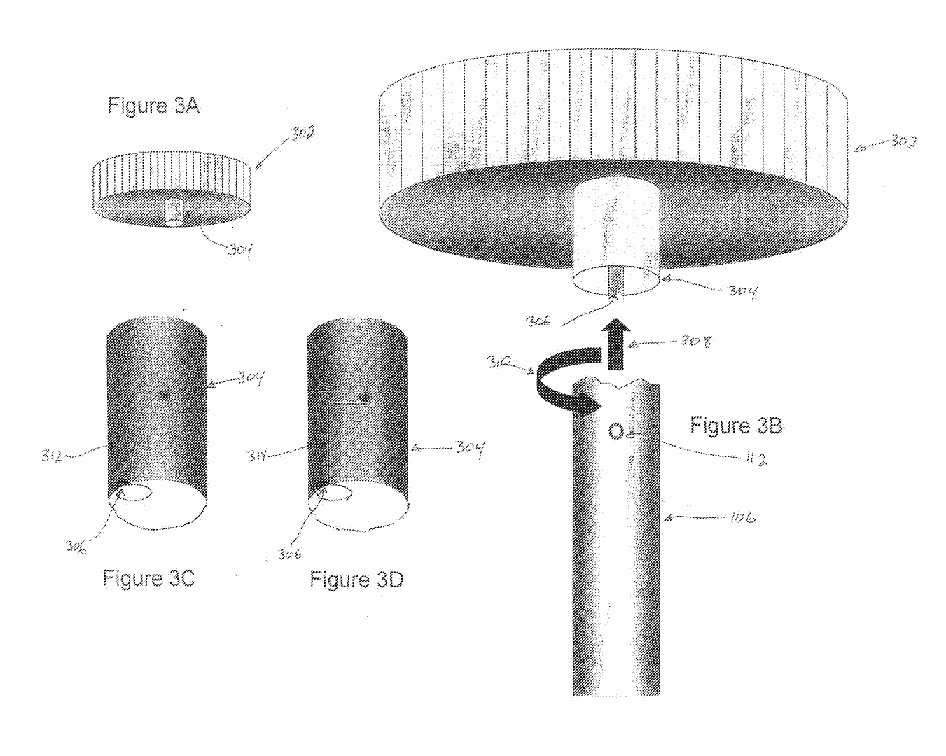

FIG. 3A shows a vial cap 302 which has a female joint 304 in the middle of its underside. The female joint 304 can be toward one side of the underside of the cap 302, as opposed to in the middle. Furthermore, as shown in FIGS. 3A and 3B, the female joint 304 extends directly downward, i.e., it makes a 90° angle with the underside surface of the cap 302. In some embodiments, the female joint 304 makes an acute angle with the underside surface of the cap 302.

As shown in FIG. 3B, once the distal portion 106 of the collection device 102 has been separated from the proximal portion 104, the distal portion 106 is attached to the underside of the vial cap 302. The distal portion 106 forms a male part which is inserted into the female part 304 on the underside of the vial cap 302.

In some embodiments, the distal portion 106 comprises at least one nub 112. In these embodiments, there is a corresponding groove 306 on the female joint 304, into which the nub 112 is inserted. Thus, in some embodiments, the distal portion 106 is inserted into the female joint 304 in the direction 308 shown in FIG. 3B, followed by a twist in the direction 310 to secure the distal portion 106 in the female joint 304. In some embodiments the groove 306 follows a curved path 312 up the female joint 304, as shown in FIG. 3C. In other embodiments, the groove 306 goes straight up the female joint 304 along the path 314 and then makes a 90° turn, as shown in FIG. 3D. In some embodiments, the paths 312 and 314 cause the distal portion 106 to turn clockwise as it is secured in the female joint 304. In other embodiments, the paths 312 and 314 cause the distal portion 106 to turn counter-clockwise as it is secured in the female joint 304.

In other embodiments, the proximal end of the distal portion 106 is a screw that is screwed in the female portion 304. In yet other embodiments, the proximal end of the distal portion 106 is a ball and the female portion 304 is a socket into which the ball fits.

In other embodiments, there is a male joint on the underside of the vial cap 302. The male joint is configured to fit into a female joint at the proximal end of the distal portion 106.

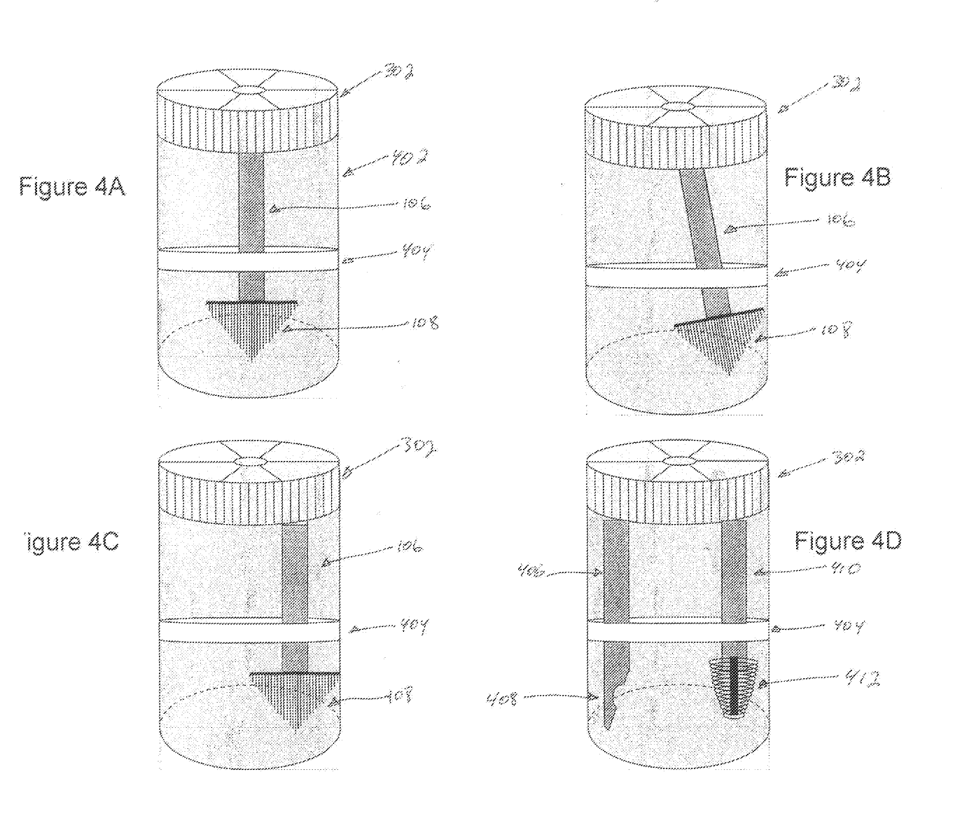

After the distal portion 106 is secured to the cap 302, the cap 302 is secured on a vial 402, FIG. 4A. The vial can contain a solution that preserves the cells for shipments to a laboratory. In one embodiment, the solution is PreservCyt® (Cytyc Corp.). In some embodiments, such as the one shown in FIG. 4A, the distal portion 106 hangs straight down from the middle of the cap 302, such that the cell collection portion 108 is below the solution surface 404. In other embodiments, the distal portion 106 hangs at an angle with respect to the cap 302, as shown in FIG. 4B. In other embodiments, as shown in FIG. 4C, the distal portion 106 hangs straight down from the side of the cap 302. In yet other embodiments, not shown, the distal portion 106 hangs at an angle with respect to the cap 302 from the side of the cap 302.

In yet other embodiments, cap 302 is configured to fit multiple cell collection devices. FIG. 4D shows an embodiment in which a spatula 408 at the end of the distal portion 406 and a brush 412 at the end of the distal portion 410 are attached to the cap 302.

In some embodiments, the cell collection portion 108, 408, or 412 remain within the solution and below the solution surface 404 even when the solution is mixed in a vortex mixer.

The embodiments shown in FIGS. 1-4 depict a cell collection device in which the shaft 114 has a circular cross section. In some embodiments, the shaft 114 has a non-circular cross section. In some of these embodiments, the cross section of shaft 114 is oval. In other embodiments, the cross section of shaft 114 is triangular. In further embodiments, the cross section of shaft 114 is a parallelogram, including a square, a rectangle, a rhombus, or a trapezoid. In yet other embodiments, the cross section of shaft 114 is polygon, such as a pentagon, a hexagon, a heptagon, or an octagon.

The invention may be embodied in other specific forms besides and beyond those described herein. The foregoing embodiments are therefore to be considered in all respects illustrative rather than limiting, and the scope of the invention is defined and limited only by the appended claims and their equivalents, rather than by the foregoing description.

1. A cell collection device, comprising
a shaft having a proximal portion, a distal portion, and a detachable joint located between the proximal and distal portions; and
a cell collector carried on a distal end of the distal portion, wherein a proximal end of the distal portion is configured for attachment to a vial cap when the proximal portion and distal portion are detached at the detachable joint.

2. The cell collection device of claim 1, wherein the detachable joint comprises a portion of the shaft having a diameter less than a diameter of the respective proximal and distal portions of the shaft.

3. The cell collection device of claim 1, wherein the detachable joint is a breakable joint.

4. The cell collection device of claim 1, wherein the detachable joint comprises a frictional interference fit between the respective proximal and distal shaft portions.

5. The cell collection device of claim 1, wherein the detachable joint comprises a nub on one of the proximal and distal shaft portions and a corresponding groove located on the other one of the proximal and distal shaft portions, the groove sized to detachably mate with the nub.

6. The cell collection device of claim 1, wherein the detachable joint comprised a threaded attachment of the proximal and distal shaft portions.

7. The cell collection device of claim 1, wherein the vial cap comprises a mechanism configured to attach to the proximal end of the distal shaft portion.

8. The cell collection device of claim 1, wherein the vial cap and distal shaft portion are configured for attachment by a friction interference fit.

9. The cell collection device of claim 1, wherein the vial cap and distal shaft portion are configured for attachment by a nub a groove assembly.

10. The cell collection device of claim 1, wherein the vial cap and distal shaft portion may be attached using a threaded joint.

11. The cell collection device of claim 1, wherein the distal shaft portion is sized such that when it is attached to a vial cap, and the vial cap is fastened to a sample vial, the cell collector is suspended within the vial.

12. The cell collection device of claim 1, wherein the distal portion of the shaft attaches to the cap at a center of the cap.

13. The cell collection device of claim 1, wherein the distal portion of the shaft attaches to the cap at an off-center location on the cap.

14. The cell collection device of claim 7, wherein the distal portion of the shaft is substantially perpendicular to an underside surface of the cap when attached thereto.

15. The cell collection device of claim 7, wherein the distal portion of the shaft attaches at an acute angle to the cap.

16. The cell collection device of claim 1, further comprising a movable sheath disposed on the shaft, wherein the sheath may be moved relative to the shaft to cover or uncover the detachable joint.

17. The cell collection device of claim 16, the shaft comprising at least one friction ridge to maintain the sheath in a position relative to the detachable joint.

18. A vial for storing a biological sample, comprising a vial cap, the cap comprising an underside, the underside comprising a mechanism configured to attach to a shaft of a cell collection device.

19. A method of collecting a biological sample with a cell collection device, the device comprising a shaft having a proximal portion, a distal portion, and a detachable joint located between the proximal and distal portions, the device further comprising and a cell collector carried on a distal end of the shaft distal portion, the method comprising:
obtaining the biological sample using the cell collector;
detaching the proximal portion of the shaft from the distal portion of the shaft at the detachable joint;
attaching the proximal end of the shaft distal portion to an underside of a vial cap; and
securing the cap and attached shaft distal portion to a vial having a solution therein, such that at least a portion of the cell collector is located below the surface of the solution.

20. The method of claim 19, wherein the shaft proximal portion is detached from the distal portion of the shaft by breaking the detachable joint.

21. The method of claim 19, wherein the proximal end of the distal portion of the shaft is attached to the underside of the vial cap by an interference fit.

22. The method of claim 19, wherein the cell collector is selected from the group of a spatula, a brush, and a cervical broom.
